(19) United States
(12) Patent Application Publication
(10) Pub. No.: US 2026/0345392 A1
(43) Pub. Date: Nov. 5, 2026

(54) OPHTHALMOLOGY ULTRAVIOLET LASER SOURCE PARAMETER MONITORING

(71) Applicant: ALCON INC., Fribourg (CH)

(72) Inventor: Matthias Foesel, Memmelsdorf (DE)

(21) Appl. No.: 19/651,241

(22) Filed: Apr. 17, 2026

Related U.S. Application Data
(60) Provisional application No. 63/797,499, filed on Apr. 30, 2025.

(51) Int. Cl. A61F 9/008

(52) U.S. Cl. CPC A61F 9/0084; A61F 2009/00844; A61F 2009/00872; A61F 2009/00885

(57) ABSTRACT

The present disclosure relates to an ophthalmic femtosecond laser system including a laser to emit a laser beam, a frequency conversion unit positioned in an optical path of the laser beam and configured to convert the laser beam to a frequency-converted laser beam, a first sensor configured to measure intensity of the laser beam, a second sensor configured to measure intensity of the frequency-converted laser beam, and a processor coupled to each of the first sensor and the second sensor. The processor is configured to execute computer-readable instructions to perform or control performance of operations that include: receiving measurements of the laser beam and the frequency-converted laser beam from the first and second sensors; calculating a conversion ratio based on the measurements from the first and second sensors; and comparing the calculated conversion ratio to a reference ratio to determine whether a performance of the femtosecond laser system has degraded.

### CROSS-REFERENCE TO RELATED APPLICATION

This application claims priority to U.S. Provisional Patent Application No. 63/797,499, filed on Apr. 30, 2025, the disclosure of which is incorporated herein by reference in its entirety.

### BACKGROUND

Many patients have visual errors associated with the refractive properties of the eye such as nearsightedness (myopia), farsightedness (hyperopia) and astigmatism. Nearsightedness can occur when light focuses before the retina, and farsightedness can occur with light refracted to a focus behind the retina. Astigmatism may occur when the corneal curvature is unequal in two or more directions. These visual impairments are commonly corrected using eyeglasses or contact lenses.

Alternatively, the cornea of the eye can be reshaped surgically to provide the desired optical correction. Eye surgery has become commonplace with some patients pursuing it as an elective procedure to avoid using contact lenses or glasses to correct refractive problems, and others pursuing it to correct adverse conditions such as cataracts. With recent developments in laser technology, laser surgery is becoming the technique of choice for ophthalmic procedures. The reason many eye surgeons prefer a surgical laser beam over manual tools like microkeratomes and forceps is that the laser beam can be focused precisely on extremely small amounts of ocular tissue, thereby enhancing accuracy and reliability of the procedure. These in turn enable better wound healing and recovery following surgery.

Different laser eye surgical systems use different types of laser beams for the various procedures and indications. These include, for instance, ultraviolet lasers, infrared lasers, and near-infrared, ultra-short pulsed lasers. Ultra-short pulsed lasers emit radiation with pulse durations as short as 10 femtoseconds and as long as 3 nanoseconds, and a wavelength between 300 nm and 3000 nm. Examples of laser systems that provide ultra-short pulsed laser beams include the iFS® Advanced Femtosecond Laser, the LenSx® Femtosecond Laser System, and the WaveLight® FS200 Femtosecond Laser System. Laser systems that provide ultra-short pulsed laser beams with durations in the femtosecond range are often referred to as femtosecond laser systems.

### SUMMARY

The present disclosure relates to an ophthalmic femtosecond laser system including a laser to emit a laser beam, a frequency conversion unit positioned in an optical path of the laser beam and configured to convert the laser beam to a frequency-converted laser beam, a first sensor configured to measure intensity of the laser beam, a second sensor configured to measure intensity of the frequency-converted laser beam, and a processor coupled to each of the first sensor and the second sensor. The processor is configured to execute computer-readable instructions to perform or control performance of operations that include: receiving measurements of the laser beam and the frequency-converted laser beam from the first and second sensors; calculating a conversion ratio based on the measurements from the first and second sensors; and comparing the calculated conversion ratio to a reference ratio to determine whether a performance of the femtosecond laser system has degraded.

### BRIEF DESCRIPTION OF THE DRAWINGS

The present disclosure relates to systems and methods for performing registration of an eye based on transition edges within the eye, wherein:

FIG. 1 is a block diagram of an example ophthalmic surgical system that may be used with and/or implement one or more embodiments of the present disclosure related to a femtosecond laser system;

FIG. 2 is a block diagram of an example femtosecond laser that may be included in the ophthalmic surgical system of FIG. 1;

FIG. 3A includes two graphs respectively representing laser beam intensity and frequency-converted laser beam intensity each as a function of power setting of a laser included in the femtosecond laser system of FIG. 2;

FIG. 3B includes two graphs respectively representing the logarithm (log) of the laser beam intensity and the log of the frequency-converted laser beam intensity each as a function of power setting of the laser included in the femtosecond laser system of FIG. 2;

FIG. 4 is a flowchart of an example method to monitor parameters of a femtosecond laser system for ophthalmology;

FIG. 5 is a flowchart of an example method to generate reference ratios and/or measurements of a femtosecond laser system for ophthalmology; and

FIG. 6 is a block diagram of an example computing system suitable for use in implementing some embodiments of the present disclosure.

### DETAILED DESCRIPTION

Some surgical approaches for reshaping the cornea include laser assisted in situ keratomileusis (hereinafter “LASIK”), photorefractive keratectomy (hereinafter “PRK”) and Small Incision Lenticular Extraction (hereinafter “SmILE,”).

LASIK and PRK are currently the two commonly performed myopia correction procedures. In the LASIK procedure, a femtosecond laser or other ultra-short pulsed laser is used to cut a conical flap that is folded open to expose the corneal stroma for photoablation with ultraviolet beams from an excimer laser. Photoablation of the corneal stroma reshapes the cornea and corrects the refractive condition such as myopia, hyperopia, astigmatism, and the like. After the corneal stroma has been photoablated with the excimer laser, the corneal flap is folded back and closed.

LASIK has the advantages of providing nearly immediate improvement in vision with a minimal amount of pain. There are, however, some disadvantages. Because a void is often created under the flap, the reshaped cornea is structurally weaker after surgery. To address this, LASIK candidates are typically limited to those patients who have corneas that are at least about 500-600 micrometers (μm) thick. Further, LASIK requires an ultra-short pulsed laser to cut the corneal flap and a separate excimer laser to ablate the corneal stroma. As would be expected, requiring multiple laser systems increases costs and requires bigger storage areas for the equipment.

If part of the cornea is removed, the pressure exerted on the cornea by the aqueous humor in the anterior chamber of the eye will act to close the void created in the cornea, resulting in a reshaped cornea. By properly selecting the size, shape, and location of a corneal void, one can obtain the desired shape, and hence, the desired optical properties of the cornea.

Another example surgical technique for refractive correction is gaining attention in certain countries and markets, and is preferred by some surgeons. Instead of ablating corneal tissue with an excimer laser following the creation of a corneal flap, some techniques involve tissue removal with two femtosecond laser incisions that intersect to create a lenticule for extraction. Lenticular extractions can be performed either with or without the creation of a corneal flap. With the flapless procedure, a refractive lenticule is created in the intact portion of the anterior cornea and removed through a small incision. For example, in a SmILE procedure, a femtosecond laser is used to make a side cut, upper surface cut and lower surface cut. A tweezer, forceps, or other tool is then used to extract the cut lens beneath the anterior surface of the cornea through the side cut.

When using femtosecond laser systems in refractive surgery, it may be desirable to achieve low energy input into the tissue. For example, flaps or lenticules may be cut with as little energy as possible. The process by which femtosecond lasers cut tissue is that of photodisruption, which involves application of high intensities to the tissue in short time periods. The photodisruption effect depends on various parameters of a laser source, including, for instance, pulse duration, beam profile, beam quality, pulse-to-pulse stability, and focusability (e.g., minimal wavefront distortions).

Some of these parameters may be measured during an incoming goods test, e.g., at or prior to the laser system being put into service such as in a factory. Measuring these parameters may require specialized tools that are not typically available in the field (e.g., in a clinic or operating room). As such, it may be difficult or even impossible in the field to quantify the quality of the laser radiation and thus cutting ability through measurement of these parameters.

During the life of a femtosecond laser system, the parameters of the laser source are not typically checked after the incoming goods test except for the power. During routine service, typically the only check is whether the femtosecond laser system is able to cut PMMA (polymethyl methacrylate) with a defined cutting energy. However, the individual parameters are not measured and it cannot be determined whether the femtosecond laser system has lost performance due to parameter degradation. In some cases, abnormalities in the femtosecond laser system are only tracked after healthcare providers (e.g., doctors) complain and then the femtosecond laser system has to be analyzed at great expense.

Some embodiments herein take advantage of the fact that the power of the laser beam generated by the laser source may depend on the parameters. Thus, some embodiments may indirectly monitor the parameters by monitoring the power of the laser beam both internal to and/or external to the laser source. For example, some embodiments may generate various power measurements of the laser beam internal and/or external to the laser source prior to and/or during treatment (e.g., surgery) and compare them to reference values, e.g., initial measurements. The initial measurements may be generated in some embodiments during the incoming goods test.

In some embodiments, a femtosecond laser system includes the laser source. The laser source includes a laser, a first sensor, and frequency conversion unit. The laser may generate a laser beam that includes radiation having a first wavelength (or wavelength range). For example, the laser beam may include infrared (IR) radiation, e.g., radiation having one or more wavelengths in the IR range. The first sensor may measure intensity of the laser beam in an optical path between the laser and the frequency conversion unit and internal to the laser source. The frequency conversion unit may convert the laser beam to a frequency-converted laser beam that includes radiation having a second wavelength (or wavelength range). For example, the frequency-converted laser beam may include ultraviolet (UV) radiation, e.g., radiation having one or more wavelengths in the UV range. The femtosecond laser system may further include a second sensor. The second sensor may measure intensity of the frequency-converted laser beam in an optical path downstream of the frequency conversion unit and external to the laser source. The intensity measurements of the laser beam and the frequency-converted laser beam and/or data derived therefrom (e.g., a ratio of one of the two measurements to the other) may be compared to reference values such as intensity measurements of the laser beam and the frequency-converted laser beam (or, e.g., a ratio thereof) generated during the incoming goods test. Insofar as some degradations in the laser source parameters may be reflected in degradations in the intensity, the measurements or data derived therefrom (e.g., a ratio) may be compared to the reference value(s) to indirectly determine parameter degradation.

Embodiments herein may alternatively or additionally monitor performance of the frequency conversion unit itself and/or alignment errors. For example, degradation of the frequency conversion unit may cause it to become less efficient at converting the laser beam to the frequency-converted laser beam, which may manifest in a reduction of intensity in the frequency-converted laser beam. By comparing a ratio of the intensity of the frequency-converted laser beam to the intensity of the laser beam against a corresponding reference value (e.g., an initial intensity at the incoming goods test of the frequency-converted laser beam to an initial intensity at the incoming goods test of the laser beam), such a reduction in intensity in the frequency-converted laser beam may be detected.

In some embodiments, the femtosecond laser system further includes a third sensor to measure intensity of the frequency-converted laser beam downstream of the second sensor. This intensity measurement and/or data derived therefrom (e.g., a ratio) may be compared to a corresponding reference value and/or to an intensity measurement or data derived therefrom corresponding to the second sensor. A misalignment of the femtosecond laser system (e.g., of the laser beam and/or frequency-converted laser beam) may be detected if, for instance, intensity as measured by the third sensor compared to that measured by the second sensor changes (e.g., is reduced).

Embodiments herein may ensure, or at least increase a likelihood, that the femtosecond laser system operates within its optimal parameters, thus maintaining the precision required for safe and effective refractive surgeries. The method also enables early detection of potential issues, allowing for timely maintenance and adjustments to be made before the system's performance is significantly compromised. This proactive approach not only enhances the safety and efficacy of the surgical procedures but also extends the operational life of the laser system, making it a cost-effective solution for medical facilities.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to ophthalmic surgical systems. However, such uses are not meant to be limiting such that the devices described may be used in any number of different contexts and applications where it may be helpful or applicable.

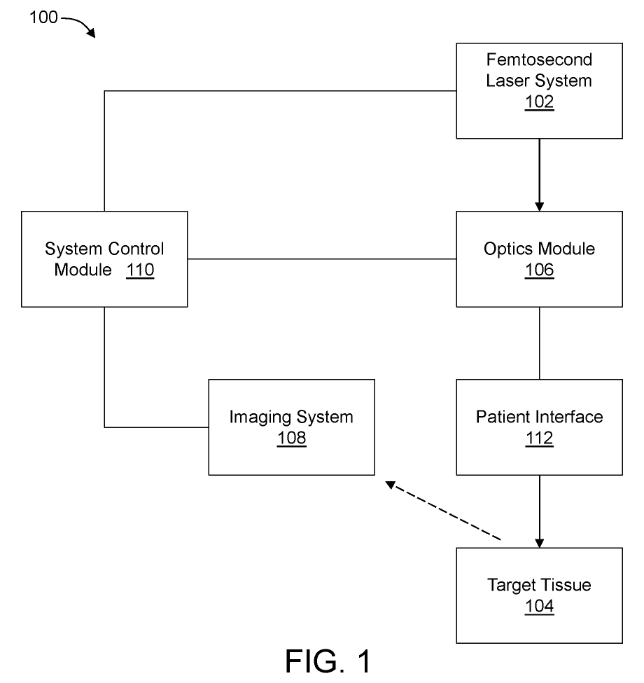

FIG. 1 is a block diagram of an example ophthalmic surgical system 100 (“system 100”) that may be used with and/or implement one or more embodiments of the present disclosure related to a femtosecond laser system 102. In general, the system 100 may be configured to perform one or more ophthalmic treatment operations with respect to performance of a procedure corresponding to a target tissue 104, which may include a portion of an eye in some embodiments. The system 100 may include one or more of the femtosecond laser system 102, an optics module 106, an imaging system 108, a system control module 110, and a patient interface 112.

The femtosecond laser system 102 may include any suitable system, apparatus, or device, configured to generate one or more laser beams that may be used to perform ophthalmic operations or tasks with respect to the target tissue 104. In some embodiments, the femtosecond laser system 102 may include multiple lasers that each generate an individual laser beam. Additionally or alternatively, the femtosecond laser system 102 may include a single laser that is configured to generate a single laser beam or multiple laser beams.

In some embodiments, the femtosecond laser system 102 may be configured to generate a pulsed laser beam that is pulsed at a high repetition rate at a pulse repetition rate of thousands of shots per second or higher with relatively low energy per pulse. For example, in some embodiments, the femtosecond laser system 102 may be a femtosecond laser that emits ultra-short pulses of light (e.g., on the order of 10^-15 seconds). Such a laser may be operated to use a relatively low energy per pulse to localize the tissue effect within the target tissue 104 that may be caused by laser-induced photodisruption by the laser beam generated by the femtosecond laser system 102. Additional details regarding an example implementation of the femtosecond laser system 102 are provided elsewhere herein.

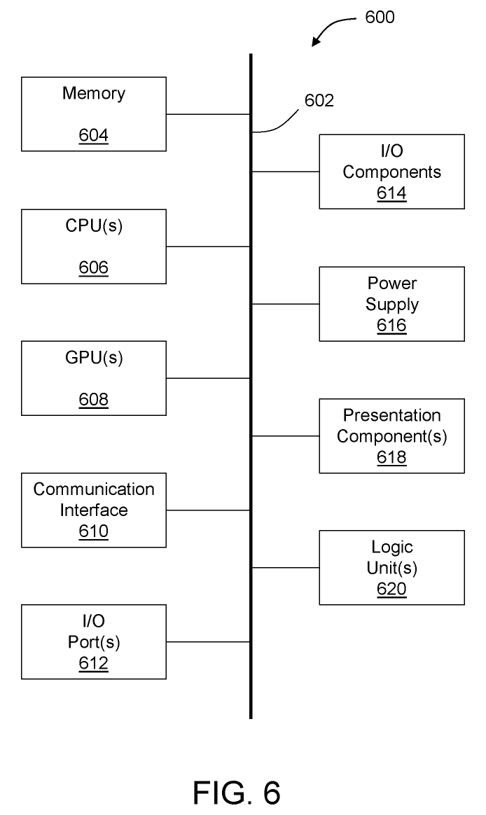

The optics module 106 may include any suitable system, apparatus, or device that may be configured to focus and direct the laser beam to the target tissue 104. For example, in some embodiments, the optics module 106 may include one or more lenses and/or one or more reflectors (e.g., mirrors). Additionally or alternatively, in some embodiments, the optics module 106 may include one or more actuators that may be configured to adjust the focusing and/or the beam direction in response to a beam control signal that may be received from the system control module 110. In these and other embodiments, the one or more actuators may be adjusted in response to a user input via any suitable user interface, such as discussed with respect to a computing system of FIG. 6. For example, in some embodiments, the user interface may include a touch screen, mouse, keyboard, joystick, foot pedal, game pad, game controller, touchscreen, etc., that may be used to provide commands for movement of the laser beam (e.g., via the actuators).

The imaging system 108 may include any suitable system, apparatus, or device, that may be configured to obtain one or more images of the eye corresponding to the target tissue 104. For example, in some embodiments, the imaging system 108 may collect reflected or scattered light or sound from the target tissue 104 to capture image data corresponding to the target tissue 104.

The imaging system 108 may include one or more different types of devices and/or systems configured to capture various different types of images of the eye corresponding to the target tissue 104. For example, in some embodiments, the imaging system 108 may include a camera configured to capture one or more camera images of the eye. In these and other embodiments, the imaging system 108 may include an ultrasound imaging device configured to capture ultrasound images of the eye. Additionally or alternatively, the imaging system 108 may include an optical coherent tomography (OCT) device configured to capture OCT images of the eye or other imaging device(s), or other imaging system or device.

The patient interface 112 may include a mount that is configured to engage with the target tissue 104 to hold the target tissue 104 in position (or to hold the patient interface 112 in position with respect to the target tissue 104) relative to the femtosecond laser system 102 during performance of the ophthalmic procedure. In these and other embodiments, the patient interface 112 may be configured to allow the laser beam to pass therethrough to allow for performance of the procedure via the laser beam.

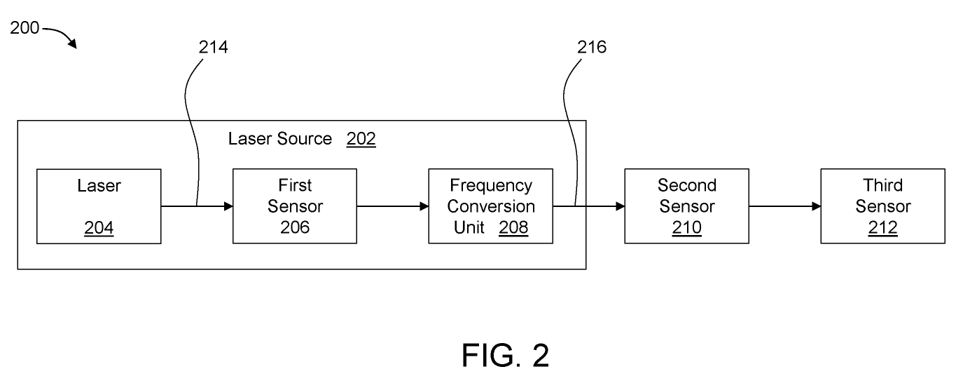

FIG. 2 is a block diagram of an example femtosecond laser system 200 that may be included in the system 100 of FIG. 1, arranged in accordance with at least one embodiment herein. The femtosecond laser system 200 may include, be included in, or otherwise correspond to the femtosecond laser system 102 of FIG. 1. In general, the femtosecond laser system 200 may include a laser source 202 with a laser 204, a first sensor 206, and a frequency conversion unit 208. The femtosecond laser system 200 may further include a second sensor 210 and/or a third sensor 212.

In general, the laser 204 may be configured to emit a laser beam 214 having a first wavelength or frequency, which is converted to a frequency-converted laser beam 216 having a second wavelength or frequency by the frequency conversion unit 208, and output for use in an ophthalmic procedure. The first sensor 206 is configured to measure intensity of the laser beam 214 while the second and third sensors 210, 212 are configured to measure intensity of the frequency-converted laser beam 216. The various measurements or data derived therefrom, such as ratios, may be compared against reference values to monitor performance of the femtosecond laser system 200. In some embodiments, a small portion of the laser beam 214 may be split off or reflected away to be measured by the first sensor 206 (e.g., by a beam splitter, a selectively reflective mirror, or other optical component). In some embodiments, less than 20%, less than 15%, less than 10%, less than 8%, less than 7%, less than 5%, or less than 4% of the laser energy of the laser beam 214 is diverted for monitoring.

In more detail, in some embodiments, the laser 204 includes a femtosecond laser capable of providing pulsed laser beams, which may be used in ophthalmic procedures, such as LASIK, SmILE, and/or other ophthalmic procedures involving localized photodisruption (e.g., laser-induced optical breakdown). Localized photodisruptions may be placed at or below the surface of a target tissue (e.g., target tissue 104 in FIG. 1) to produce high-precision material processing. For example, a micro-optics scanning system (not illustrated in FIG. 2) may be used to scan the pulsed laser beam to produce an incision in the target tissue and form three-dimensional removable structures of the target tissue. The term “scan” or “scanning” refers to the movement of the focal point of the pulsed laser beam along a desired path or in a desired pattern.

An example set of parameters for the laser beam 214 emitted by such a femtosecond laser may include a pulse duration of 50 femtoseconds to 10 picoseconds, a pulse frequency of 10 kilohertz (KHz) to 20 megahertz (MHz), a wavelength of 100 nanometers (nm) to 2000 nm (e.g., 1030 nm to 1064 nm or other range of IR wavelengths), and a pulse energy of 50 nanojoules to 5 microjoules.

The frequency conversion unit 208 may include one or more crystals and/or other components which, in response to input in the form of the laser beam 214, output the frequency-converted laser beam 216. In some embodiments, the frequency conversion unit 208 may include a first conversion crystal and a second conversion crystal located after the first conversion crystal in a direction of the laser beam 214 propagation. The first conversion crystal may double portions of a fundamental wavelength of the laser beam 214 in their frequency. The second conversion crystal may cause a frequency mixture in an interaction of the fundamental wavelength and the components with frequency-doubled wavelength, which may result in a new wavelength. As an example, the laser beam 214 may have a fundamental wavelength of approximately 1040 nanometers (nm) which may be partially converted to 532 nm by the first conversion crystal; frequency mixing by the second conversion crystal of remaining 1040 nm fundamental wavelength portions with the 532 nm doubled portions may produce radiation with a wavelength of 347 nm as the frequency-converted laser beam 216.

The frequency-converted laser beam 216 may include UV pulses capable of photodisrupting one or more intraocular targets within the eye (e.g., the target tissue 104 of FIG. 1). An example set of parameters for the frequency-converted laser beam 216 output from the frequency conversion unit 208 may be the same as, similar to, or different than those of the laser beam 216. For example, the frequency-converted laser beam 216 may have a pulse duration of 100 picoseconds to 5 nanoseconds (e.g., 0.7 ns), a pulse frequency of 10 KHz to 500 KHz, a wavelength of about 355 nm, and a pulse energy of 160 nanojoules to 5 microjoules.

The frequency conversion unit 208 may be characterized by non-linear behavior. For example, the frequency-converted laser beam 216 intensity may be exponentially related to the laser beam 214 intensity. The laser beam 214 intensity may linearly depend on the power setting of the laser 204 such that as the power setting is increased, the laser beam 214 intensity increases linearly. Because the laser beam 214 intensity linearly depends on the power setting and the frequency-converted laser beam 216 intensity exponentially depends on the laser beam 214 intensity, the frequency-converted laser beam 216 intensity exponentially depends on the power setting.

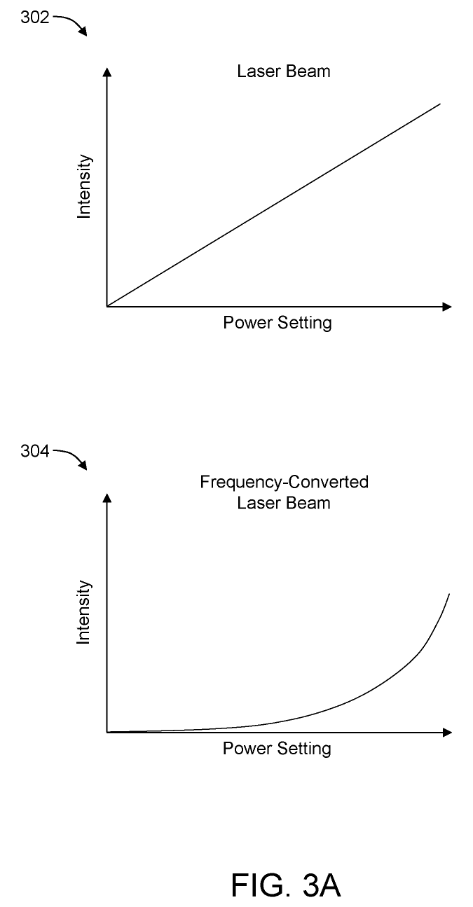

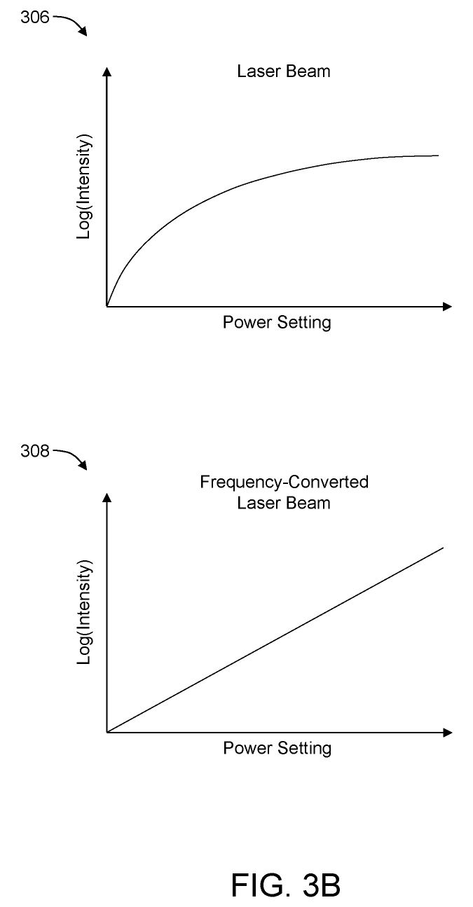

FIG. 3A includes two graphs 302, 304 respectively representing laser beam 214 intensity and frequency-converted laser beam 216 intensity each as a function of power setting of the laser 204, arranged in accordance with at least one embodiment herein. As illustrated in the graph 302 of FIG. 3A, the laser beam 214 intensity linearly depends on the power setting. As illustrated in the graph 304 of FIG. 3A, the frequency-converted laser beam 214 intensity exponentially depends on the power setting.

FIG. 3B includes two graphs 306, 308 respectively representing the logarithm (log) of the laser beam 214 intensity and the log of the frequency-converted laser beam 216 intensity each as a function of power setting of the laser 204, arranged in accordance with at least one embodiment herein. As illustrated in the graph 306 of FIG. 3B, the log of the laser beam 214 intensity logarithmically depends on the power setting. As illustrated in the graph 308 of FIG. 3B, the log of the frequency-converted laser beam 216 intensity linearly depends on the power setting.

Due to the different dependency of the laser beam 214 intensity and the frequency-converted laser beam 216 intensity on the power setting, a value (e.g., a ratio) may be generated from these variables (laser beam 214 intensity and frequency-converted laser beam 216 intensity) which also changes when one of the two variables changes. In comparison, if the frequency conversion unit 208 behaved linearly, the ratio may remain constant when one of the variables changes.

Returning to FIG. 2, although the femtosecond laser system 200 may be used to photoalter a variety of materials (e.g., organic, inorganic, or a combination thereof), the femtosecond laser system 200 may be suitable for ophthalmic applications in some embodiments. In these cases, focusing optics (e.g., optics module 106 in FIG. 1) may direct the frequency-converted laser beam 216 toward an eye (for example, onto or into a cornea) for treatment of corneal tissue. In these and other embodiments, the femtosecond laser system 200 may be part of an ophthalmic surgical system, such as the system 100 of FIG. 1, that also includes a patient interface (e.g., patient interface 112) contacting the eye to, e.g., change the shape of the cornea (for example, flatten or curve) prior to scanning the frequency-converted laser beam 216 toward the eye and/or to fix the position and orientation of the eye relative to the laser system 200.

Although not illustrated in FIG. 2, the femtosecond laser system 200 may further include or be part of a system that includes one or more other components, devices, or systems. For example, an energy control module may be provided to vary the pulse energy of the laser beam 214 (and thereby of the frequency-converted laser beam 216), a Z-scanner to modify a depth of the laser beam 214 (and thereby of the frequency-converted laser beam 216), a controller (e.g., the system control module 110), a prism (e.g., a Dove or Pechan prism, or the like, as or as part of the optics module 106), and an XY-scanner to deflect or direct the frequency-converted laser beam 216 from the laser source 202 on or within target tissue (e.g., the target tissue 104). The controller, such as a processor operating suitable control software, may be operatively coupled with the Z-scanner, the XY-scanner, and/or the energy control unit to direct a scan line of the frequency-converted laser beam 216 along a scan pattern on or in the target tissue. In this and other embodiments, the femtosecond system may further include or be part of a system that includes a beam splitter and a detector (e.g., as or as part of the imaging system 108) coupled to the controller for a feedback control mechanism (not shown) of the frequency-converted laser beam 216. Other feedback methods may also be used, including but not necessarily limited to a position encoder on the scanner, or the like. In this and other embodiments, the pattern of pulses may be summarized in machine readable data of tangible storage media in the form of a treatment table. A treatment table may be adjusted according to, e.g., feedback input into the controller from an imaging system, such as the imaging system 108 of FIG. 1. Optionally, the feedback may be manually entered into the controller by a system operator. The feedback may also be provided by integrating a wavefront measurement system (not shown) with the femtosecond laser system 200 and/or a system that includes the femtosecond laser system 200. The controller may continue and/or terminate a corneal tissue treatment in response to the feedback, and may also modify the planned treatment based at least in part on the feedback.

In operation, the laser emits the laser beam 214. The frequency conversion unit 208 is positioned in an optical path of the laser beam 214 and converts the laser beam 214 to the frequency-converted laser beam 216. The first sensor 206 measures intensity of the laser beam 214. The second sensor 210 measures intensity of the frequency-converted laser beam 216.

A processor or controller, such as may be included in the system control module 110, may be coupled to the first sensor 206 and the second sensor 210. The processor may be configured to execute computer-readable instructions to perform or control performance of operations. The operations may include receiving measurements of the laser beam 214 and the frequency-converted laser beam 216 from the first and second sensors 206, 210. The operations may include calculating a conversion ratio based on the measurements from the first and second sensors 206, 210. The operations may include comparing the calculated conversion ratio to a reference ratio (e.g., as determined during manufacturing or validation of the femtosecond laser system 200) to determine whether a performance of the femtosecond laser system 200 has degraded.

As illustrated in FIG. 2, the femtosecond laser system 200 may optionally further include the third sensor 212, which may be coupled to the processor, and which may measure intensity of the frequency-converted laser beam 216 downstream of the second sensor 210. The operations executed by the processor may further include receiving measurements of the frequency-converted laser beam 216 from the third sensor 212, calculating a second conversion ratio based on the measurements from the first and third sensors 206, 212, and comparing the calculated second conversion ratio to the calculated conversion ratio to determine whether at least one of: the femtosecond laser system 200 has a desired alignment, the second sensor 210 is experiencing degraded performance, or the third sensor 212 is experiencing degraded performance. For example, assume the conversion ratio is the ratio of the second sensor 210 measurement to the first sensor 206 measurement and the second conversion ratio is the ratio of the third sensor 212 measurement to the first sensor 206 measurement. If the second conversion ratio is less than the conversion ratio, it may be determined that the femtosecond laser system 200 is misaligned (lacks a desired alignment) as the intensity is decreasing as the frequency-converted laser beam 216 progresses further downstream, or it may be determined that the third sensor 212 is experiencing degraded performance. Alternatively, it may be determined that the second sensor 210 is experiencing degraded performance if the conversion ratio is less than the second conversion ratio.

In some embodiments, the femtosecond laser system 200 may monitor the first conversion ratio (e.g., the ratio between the first sensor 206 and the second sensor 210) normally, or at a certain periodic interval, and may monitor, check, or validate the second conversion ratio (e.g., the ratio between the first sensor 206 and the third sensor 212) less frequently. For example, the second conversion ratio may be monitored, checked, or validated every other time the first conversion ratio is checked, every third, every fifth, every tenth, or every hundredth, etc. time the first conversion ratio is checked. As another example, the second conversion ratio may be monitored, checked, or validated once per given unit time, such as once per every 5 minutes, 10 minutes, 30 minutes, hour, 2 hours, 4 hours, 8 hours, twice per day, once per day, every other day, once per week, etc. For example, the first conversion ratio may be used to monitor the actual laser itself, and the second conversion ratio may be used to monitor the monitoring system (e.g., the sensors and associated processing), and as such, may be checked less frequently.

Although not depicted in FIG. 2, the femtosecond laser system 200 may further include or be coupled to an alert system that may also be coupled to the processor. The alert system may generate an alert in response to a determination that the performance of the femtosecond laser system 200 has degraded beyond a first threshold. The processor may determine that the performance has degraded beyond the first threshold if, for example, the conversion ratio deviates from the reference ratio by an amount in excess of the first threshold. The operations may further include automatically powering down the laser in response to a determination that the performance of the femtosecond laser system has degraded beyond a second threshold that is past the first threshold. The processor may determine that the performance has degraded beyond the second threshold if, for example, the conversion ratio deviates from the reference ratio by an amount in excess of the second threshold.

The reference ratio may be stored in a memory coupled to the processor and may be based on initial measurements from the first and second sensors generated at a previous time, such as at or during an incoming goods test. The operations may further include generating multiple reference ratios at the previous time, including iteratively setting power of the laser to a different power, and, while at each power: receiving measurements of the laser beam and the frequency converted laser beam from the first and second sensors while the power of the laser is set at a corresponding power; calculating a reference ratio for the corresponding power based on the measurements from the first and second sensors; and storing the reference ratio for the corresponding power in the memory.

Although not depicted in FIG. 2, the femtosecond laser system 200 may further include or be part of a system that includes a data storage device coupled to the processor. The operations may further include, each time the femtosecond laser system is used in an ophthalmic procedure and/or at one or more other times, logging at least one of measurements from the first, second, and/or third sensors 206, 210, 212 and/or a corresponding calculated conversion ratio in the data storage device. The operations may further include analyzing logged data in the data storage device for trend analysis to monitor degradation of the femtosecond laser system 200 over time.

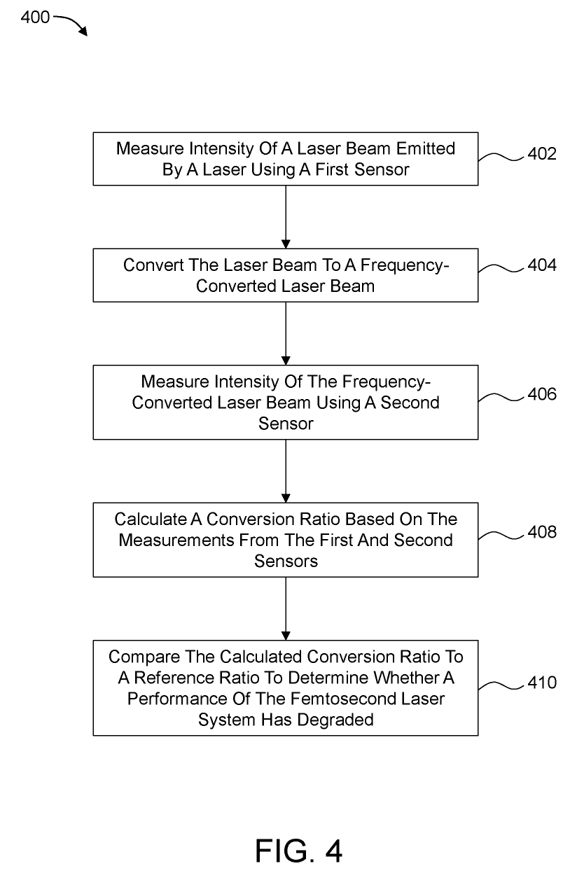

FIG. 4 is a flowchart of an example method 400 to monitor parameters of a femtosecond laser system for ophthalmology, arranged in accordance with at least one embodiment herein. The method 400 may be performed or controlled by any suitable system, apparatus, or device. For example, the method 400 may be implemented, in whole or in part, in the ophthalmic surgical system 100 of FIG. 1, the femtosecond laser system 200 of FIG. 2, or other system or device herein, and/or using the control module 110 of FIG. 1 or other processor, control or computing system herein. Alternatively or additionally, the method 400 may be embodied in computer-readable instructions stored on a computer-readable storage medium, the computer-readable instructions executable by a processor to perform or control performance of the method 400. The method 400 may include one or more of blocks 402, 404, 406, 408, and/or 410. Although illustrated with discrete blocks, the steps and operations associated with one or more blocks of the method 400 may be divided into additional blocks,combined into fewer blocks, or eliminated, depending on the particular implementation.

At block 402, the method 400 may include measuring intensity of a laser beam emitted by a laser using a first sensor. For example, block 402 may include the first sensor 206 of FIG. 2 measuring intensity of the laser beam 214 emitted by the laser 204. Block 402 may be followed by block 404.

At block 404, the method 400 may include converting the laser beam to a frequency-converted laser beam. For example, block 404 may include the frequency conversion unit 208 converting the laser beam 214 to the frequency-converted laser beam 216. In some embodiments, converting the laser beam to the frequency-converted laser beam may include converting infrared radiation to ultraviolet radiation. Block 404 may be followed by block 406.

At block 406, the method 400 may include measuring intensity of the frequency-converted laser beam using a second sensor. For example, block 406 may include the second sensor 210 of FIG. 2 measuring intensity of the frequency-converted laser beam 216. Block 406 may be followed by block 408.

At block 408, the method 400 may include calculating a conversion ratio based on the measurements from the first and second sensors. Calculating the conversion ratio at block 408 may include calculating a ratio of the measurement from the second sensor 210 of the intensity of the frequency-converted laser beam 216 to the measurement from the first sensor 206 of the intensity of the laser beam 214. Alternatively, calculating the conversion ratio at block 408 may include calculating a ratio of the measurement from the first sensor 206 of the intensity of the laser beam 214 to the measurement from the second sensor 210 of the intensity of the frequency-converted laser beam 216. Block 408 may be followed by block 410.

At block 410, the method 400 may include comparing the calculated conversion ratio to a reference ratio to determine whether a performance of the femtosecond laser system has degraded. The reference ratio may include the same ratio as the conversion ratio calculated at a previous time (e.g., during an incoming goods test) for a same power setting of the laser. The reference ratio may serve as a baseline against which the current and future conversion ratios at the same power setting may be compared to monitor performance and degradation of the femtosecond laser system.

In some embodiments, the method 400 may further include measuring intensity of the frequency-converted laser beam using a third sensor, such as the third sensor 212 of FIG. 2, downstream of the second sensor. The method 400 may further include calculating a second conversion ratio based on the measurements from the first and third sensors. Calculating the second conversion ratio may include calculating a ratio of the measurement from the third sensor 212 of the intensity of the frequency-converted laser beam 216 to the measurement from the first sensor 206 of the intensity of the laser beam 214. Alternatively, calculating the conversion ratio at block 408 may include calculating a ratio of the measurement from the first sensor 206 of the intensity of the laser beam 214 to the measurement from the third sensor 212 of the intensity of the frequency-converted laser beam 216. The method 400 may further include comparing the calculated second conversion ratio to the calculated conversion ratio to determine whether at least one of: the femtosecond laser system has a desired alignment, the second sensor is experiencing degraded performance, or the third sensor is experiencing degraded performance.

In some embodiments, the method 400 may further include generating an alert in response to a determination that the performance of the femtosecond laser system has degraded beyond a first threshold. Alternatively or additionally, the method 400 may further include automatically powering down the laser in response to a determination that the performance of the femtosecond laser system has degraded beyond a second threshold that is past the first threshold.

In some embodiments, the method 400 may further include, each time the femtosecond laser system is used in an ophthalmic procedure, logging at least one of measurements from the first and second sensors or a corresponding calculated conversion ratio. Alternatively or additionally, the method 400 may further include analyzing logged data for trend analysis to monitor degradation of the femtosecond laser system over time.

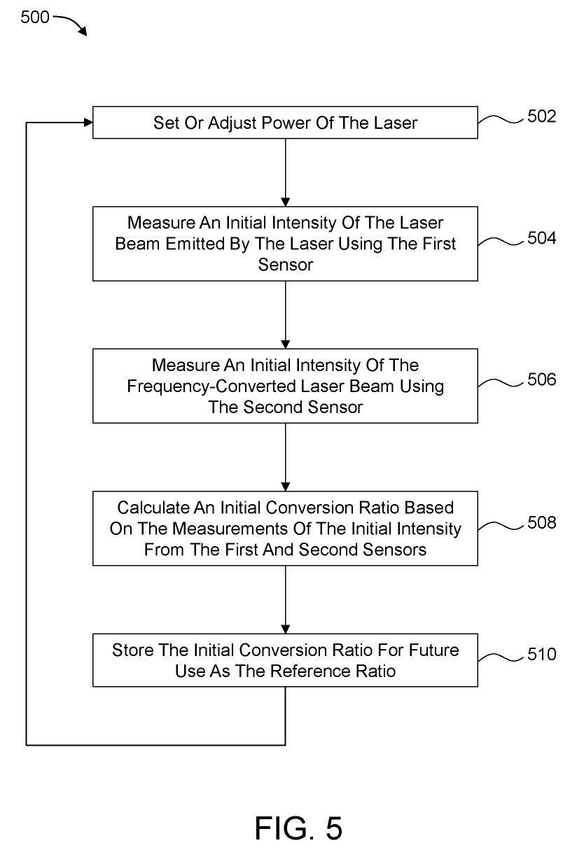

FIG. 5 is a flowchart of an example method 500 to generate reference ratios and/or measurements of a femtosecond laser system for ophthalmology, arranged in accordance with at least one embodiment herein. The method 500 may be performed, e.g., during an incoming goods test and/or in a factory prior to the femtosecond laser system being deployed for performing ophthalmic procedures. The method 500 may collect initial measurement data (e.g., measurements and/or ratios or other information derived from the measurements) for multiple different laser power settings that may serve as a baseline against which future measurement data may be compared to monitor performance and degradation of the femtosecond laser system. Some or all of the method 500 of FIG. 5 may be combined with some or all of the method 400 of FIG. 4 or other methods or operations described herein.

The method 500 may be performed or controlled by any suitable system, apparatus, or device. For example, the method 500 may be implemented, in whole or in part, in the ophthalmic surgical system 100 of FIG. 1, the femtosecond laser system 200 of FIG. 2, or other system or device herein, and/or using the control module 110 of FIG. 1 or other processor, control or computing system herein. Alternatively or additionally, the method 500 may be embodied in computer-readable instructions stored on a computer-readable storage medium, the computer-readable instructions executable by a processor to perform or control performance of the method 500. The method 500 may include one or more of blocks 502, 504, 506, 508, and/or 510. Although illustrated with discrete blocks, the steps and operations associated with one or more blocks of the method 500 may be divided into additional blocks,combined into fewer blocks, or eliminated, depending on the particular implementation.

As previously indicated, the method 500 may be performed during an incoming goods test and/or in a factory prior to the femtosecond laser system being deployed in the field to perform ophthalmic procedures. In general, the method 500 may involve iteratively setting or adjusting a power of a laser of the femtosecond laser system to a given power at block 502 and performing blocks 504, 506, 508, and 510 while the laser is set at the given power.

In more detail, at block 502, the method 500 may include setting a power of the laser. Block 502 may be followed by block 504.

At block 504, the method 500 may include measuring an initial intensity of the laser beam emitted by the laser using the first sensor. For example, block 504 may include the first sensor 206 of FIG. 2 measuring the initial intensity of the laser beam 214 emitted by the laser 204. Block 504 may be followed by block 506.

At block 506, the method 500 may include measuring an initial intensity of the frequency-converted laser beam using the second sensor. For example, block 506 may include the second sensor 210 of FIG. 2 measuring the initial intensity of the frequency-converted laser beam 216 emitted by the laser 204. Block 506 may be followed by block 508.

At block 508, the method 500 may include calculating an initial conversion ratio based on the measurements of the initial intensity from the first and second sensors. Calculating the initial conversion ratio (corresponding to the power set at block 502) at block 508 may include calculating a ratio of the measurement from the second sensor 210 of the initial intensity of the frequency-converted laser beam 216 to the measurement from the first sensor 206 of the initial intensity of the laser beam 214. Alternatively, calculating the conversion ratio (corresponding to the power set at block 502) at block 508 may include calculating a ratio of the measurement from the first sensor 206 of the initial intensity of the laser beam 214 to the measurement from the second sensor 210 of the initial intensity of the frequency-converted laser beam 216. Block 508 may be followed by block 510.

At block 510, the method 500 may include storing the initial conversion ratio (e.g., as determined at block 508) for future use as the reference ratio. Block 510 may be followed by block 502 if there is at least one other power setting for which initial measurements are desired. If so, at block 502, the method 500 may include adjusting the power of the laser to a different power, and then repeating blocks 504, 506, 508, 510 at the different power. If not, the method 500 may terminate.

While the method 500 is described with reference to the first sensor and the second sensor (e.g., the first sensor 206 and the second sensor 210), it will be appreciated that the same approach may be undertaken using the first sensor and the third sensor (e.g., the first sensor 206 and the third sensor 212) to store, determine, or otherwise obtain a reference ratio between the first sensor and the third sensor.

FIG. 6 is a block diagram of an example computing system 600 suitable for use in implementing some embodiments of the present disclosure. Computing system 600 may include, be included in, or be coupled to the system control module 110 of FIG. 1, or the processor, or other control systems discussed herein. Computing system 600 may include an interconnect system 602 that directly or indirectly couples the following devices: memory 604, one or more central processing units (CPUs) 606, one or more graphics processing units (GPUs) 608, a communication interface 610, I/O ports 612, input/output components 614, a power supply 616, one or more presentation components 618 (e.g., display(s)), and one or more logic units 620.

Although the various blocks of FIG. 6 are shown as connected via the interconnect system 602 with lines, this is not intended to be limiting and is for clarity only. For example, in some embodiments, a presentation component 618, such as a display device, may be considered an I/O component 614 (e.g., if the display is a touch screen). As another example, the CPUs 606 and/or GPUs 608 may include memory (e.g., the memory 604 may be representative of a storage device in addition to the memory of the GPUs 608, the CPUs 606, and/or other components). In other words, the computing system of FIG. 6 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 6.

The interconnect system 602 may represent one or more links or busses, such as an address bus, a data bus, a control bus, or a combination thereof. The interconnect system 602 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 606 may be directly connected to the memory 604. Further, the CPU 606 may be directly connected to the GPU 608. Where there is direct, or point-to-point, connection between components, the interconnect system 602 may include a PCIe link to carry out the connection. In these examples, a PCI bus need not be included in the computing system 600.

The memory 604 may include any of a variety of computer-readable media. The computer-readable media may be any available media that may be accessed by the computing system 600. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 604 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 600. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 606 may be configured to execute at least some of the computer-readable instructions to control one or more components of the computing system 600 (or other devices or systems to which the computing system 600 may be coupled) to perform one or more of the methods and/or processes described herein. The CPU(s) 606 may each include one or more cores (e.g., one, two, four, eight, twenty-eight, seventy-two, etc.) that are capable of handling a multitude of software threads simultaneously. The CPU(s) 606 may include any type of processor, and may include different types of processors depending on the type of computing system 600 implemented (e.g., processors with fewer cores for mobile devices and processors with more cores for servers). For example, depending on the type of computing system 600, the processor may be an Advanced RISC Machines (ARM) processor implemented using Reduced Instruction Set Computing (RISC) or an x86 processor implemented using Complex Instruction Set Computing (CISC). The computing system 600 may include one or more CPUs 606 in addition to one or more microprocessors or supplementary co-processors, such as math co-processors.

In addition to or alternatively from the CPU(s) 606, the GPU(s) 608 may be configured to execute at least some of the computer-readable instructions to control one or more components of the computing system 600 (or other devices or systems to which the computing system 600 may be coupled) to perform one or more of the methods and/or processes described herein. One or more of the GPU(s) 608 may be an integrated GPU (e.g., with one or more of the CPU(s) 606 and/or one or more of the GPU(s) 608 may be a discrete GPU. In embodiments, one or more of the GPU(s) 608 may be a coprocessor of one or more of the CPU(s) 606. The GPU(s) 608 may be used by the computing system 600 to render graphics (e.g., 10D graphics) or perform general purpose computations. For example, the GPU(s) 608 may be used for General-Purpose computing on GPUs (GPGPU). The GPU(s) 608 may include hundreds or thousands of cores that are capable of handling hundreds or thousands of software threads simultaneously. The GPU(s) 608 may generate pixel data for output images in response to rendering commands (e.g., rendering commands from the CPU(s) 606 received via a host interface). The GPU(s) 608 may include graphics memory, such as display memory, for storing pixel data or any other suitable data, such as GPGPU data. The display memory may be included as part of the memory 604. The GPU(s) 608 may include two or more GPUs operating in parallel (e.g., via a link). The link may directly connect the GPUs (e.g., using NVLINK) or may connect the GPUs through a switch (e.g., using NVSwitch). When combined together, each GPU 608 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 606 and/or the GPU(s) 608, the logic unit(s) 620 may be configured to execute at least some of the computer-readable instructions to control one or more components of the computing system 600 (or other devices or systems to which the computing system 600 may be coupled) to perform one or more of the methods and/or processes described herein. In embodiments, the CPU(s) 606, the GPU(s) 608, and/or the logic unit(s) 620 may discretely or jointly perform any combination of the methods, processes and/or portions thereof. One or more of the logic units 620 may be part of and/or integrated in one or more of the CPU(s) 606 and/or the GPU(s) 608 and/or one or more of the logic units 620 may be discrete components or otherwise external to the CPU(s) 606 and/or the GPU(s) 608. In embodiments, one or more of the logic units 620 may be a coprocessor of one or more of the CPU(s) 606 and/or one or more of the GPU(s) 608.

Examples of the logic unit(s) 620 include one or more processing cores and/or components thereof, such as Tensor Cores (TCs), Tensor Processing Units(TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 610 may include one or more receivers, transmitters, and/or transceivers that enable the computing system 600 to communicate with other computing systems via an electronic communication network, including wired and/or wireless communications. The communication interface 610 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 612 may enable the computing system 600 to be logically coupled to other devices including the I/O components 614, the presentation component(s) 618, and/or other components, some of which may be built into (e.g., integrated in) the computing system 600. Illustrative I/O components 614 include a microphone, mouse, keyboard, joystick, game pad, game controller, satellite dish, scanner, printer, wireless device, etc. The I/O components 614 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing system 600. The computing system 600 may include depth cameras, such as stereoscopic camera systems, infrared camera systems, RGB camera systems, touchscreen technology, and combinations of these, for gesture detection and recognition. Additionally, the computing system 600 may include accelerometers or gyroscopes (e.g., as part of an inertia measurement unit (IMU)) that enable detection of motion. In some examples, the output of the accelerometers or gyroscopes may be used by the computing system 600 to render immersive augmented reality or virtual reality.

The power supply 616 may include a hard-wired power supply, a battery power supply, or a combination thereof. The power supply 616 may provide power to the computing system 600 to enable the components of the computing system 600 to operate.

The presentation component(s) 618 may include a display (e.g., a monitor, a touch screen, a television screen, a heads-up-display (HUD), other display types, or a combination thereof), speakers, and/or other presentation components. The presentation component(s) 618 may receive data from other components (e.g., the GPU(s) 608, the CPU(s) 606, etc.), and output the data (e.g., as an image, video, sound, etc.).

Modifications, additions, or omissions may be made to FIG. 6 without departing from the scope of the present disclosure. For example, the computing system 600 may include more or fewer elements depending on the implementation. Further, the computing system 600 may be configured to perform any number of operations as compared to those explicitly described.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example The following is a non-limiting summary of some example implementations presented herein.

Example 1. A femtosecond laser system for ophthalmology, comprising:
a laser configured to emit a laser beam;
a frequency conversion unit positioned in an optical path of the laser beam and configured to convert the laser beam to a frequency-converted laser beam;
a first sensor configured to measure intensity of the laser beam;
a second sensor configured to measure intensity of the frequency-converted laser beam; and
a processor coupled to each of the first sensor and the second sensor, the processor configured to execute computer-readable instructions to perform or control performance of operations comprising:
receiving measurements of the laser beam and the frequency-converted laser beam from the first and second sensors;
calculating a conversion ratio based on the measurements from the first and second sensors; and
comparing the calculated conversion ratio to a reference ratio to determine whether a performance of the femtosecond laser system has degraded.

Example 2. The femtosecond laser system of any example herein, particularly of example 1, further comprising a third sensor coupled to the processor and configured to measure intensity of the frequency-converted laser beam downstream of the second sensor.

Example 3. The femtosecond laser system of any example herein, particularly of example 2, wherein the operations further comprise:
receiving measurements of the frequency-converted laser beam from the third sensor;
calculating a second conversion ratio based on the measurements from the first and third sensors; and
comparing the calculated second conversion ratio to the calculated conversion ratio to determine whether at least one of: the femtosecond laser system has a desired alignment, the second sensor is experiencing degraded performance, or the third sensor is experiencing degraded performance.

Example 4. The femtosecond laser system of any example herein, particularly any one of examples 1-3, further comprising an alert system coupled to the processor and configured to generate an alert in response to a determination that the performance of the femtosecond laser system has degraded beyond a first threshold.

Example 5. The femtosecond laser system of any example herein, particularly of example 4, wherein the operations further comprise automatically powering down the laser in response to a determination that the performance of the femtosecond laser system has degraded beyond a second threshold that is past the first threshold.

Example 6. The femtosecond laser system of any example herein, particularly any one of examples 1-5, wherein:
the laser beam comprises infrared radiation;
the frequency-converted laser beam comprises ultraviolet radiation; and
the frequency conversion unit is configured to convert infrared radiation to ultraviolet radiation.

Example 7. The femtosecond laser system of any example herein, particularly any one of examples 1-6, wherein the reference ratio is stored in a memory coupled to the processor and is based on initial measurements from the first and second sensors generated at a previous time.

Example 8. The femtosecond laser system of any example herein, particularly of example 7, wherein the operations further comprise generating a plurality of reference ratios at the previous time, including iteratively setting power of the laser to a different power, and, while at each power:
receiving measurements of the laser beam and the frequency converted laser beam from the first and second sensors while the power of the laser is set at a corresponding power;
calculating a reference ratio for the corresponding power based on the measurements from the first and second sensors; and
storing the reference ratio for the corresponding power in the memory.

Example 9. The femtosecond laser system of any example herein, particularly any one of examples 1-8, further comprising a data storage device coupled to the processor, wherein the operations further comprise, each time the femtosecond laser system is used in an ophthalmic procedure, logging at least one of measurements from the first and second sensors or a corresponding calculated conversion ratio in the data storage device.

Example 10. The femtosecond laser system of any example herein, particularly of example 9, wherein the operations further comprise, analyzing logged data in the data storage device for trend analysis to monitor degradation of the femtosecond laser system over time.

Example 11. A method to monitor parameters of a femtosecond laser system for ophthalmology, the method comprising:
measuring intensity of a laser beam emitted by a laser using a first sensor;
converting the laser beam to a frequency-converted laser beam;
measuring intensity of the frequency-converted laser beam using a second sensor;
calculating a conversion ratio based on the measurements from the first and second sensors; and
comparing the calculated conversion ratio to a reference ratio to determine whether a performance of the femtosecond laser system has degraded.

Example 12. The method of any example herein, particularly of example 11, further comprising:
measuring intensity of the frequency-converted laser beam using a third sensor downstream of the second sensor;
calculating a second conversion ratio based on the measurements from the first and third sensors; and
comparing the calculated second conversion ratio to the calculated conversion ratio to determine whether at least one of: the femtosecond laser system has a desired alignment, the second sensor is experiencing degraded performance, or the third sensor is experiencing degraded performance.

Example 13. The method of any example herein, particularly any one of examples 11-12, further comprising, generating an alert in response to a determination that the performance of the femtosecond laser system has degraded beyond a first threshold.

Example 14. The method of any example herein, particularly of example 13, further comprising, automatically powering down the laser in response to a determination that the performance of the femtosecond laser system has degraded beyond a second threshold that is past the first threshold.

Example 15. The method of any example herein, particularly any one of examples 11-14, wherein converting the laser beam to a frequency-converted laser beam comprises converting infrared radiation to ultraviolet radiation.

Example 16. The method of any example herein, particularly any one of examples 11-15, further comprising, at a previous time:
measuring an initial intensity of the laser beam emitted by the laser using the first sensor;
measuring an initial intensity of the frequency-converted laser beam using the second sensor;
calculating an initial conversion ratio based on the measurements of the initial intensity from the first and second sensors; and
storing the initial conversion ratio for future use as the reference ratio.

Example 17. The method of any example herein, particularly of example 16, further comprising, generating a plurality of reference ratios at the previous time, including iteratively setting power of the laser to a different power, and, while at each power:
receiving measurements of the laser beam and the frequency converted laser beam from the first and second sensors while the power of the laser is set at a corresponding power;
calculating a reference ratio for the corresponding power based on the measurements from the first and second sensors; and
storing the reference ratio for the corresponding power for future use.

Example 18. The method of any example herein, particularly any one of examples 11-17, further comprising, each time the femtosecond laser system is used in an ophthalmic procedure, logging at least one of measurements from the first and second sensors or a corresponding calculated conversion ratio.

Example 19. The method of any example herein, particularly of example 18, further comprising, analyzing logged data for trend analysis to monitor degradation of the femtosecond laser system over time.

Example 20. A non-transitory computer-readable storage medium comprising computer-readable instructions executable by a processor to perform or control performance of operations comprising:
measuring intensity of a laser beam emitted by a laser using a first sensor;
converting the laser beam to a frequency-converted laser beam;
measuring intensity of the frequency-converted laser beam using a second sensor;
calculating a conversion ratio based on the measurements from the first and second sensors; and
comparing the calculated conversion ratio to a reference ratio to determine whether a performance of a femtosecond laser system that includes the laser, the first sensor, and the second sensor has degraded.

What is claimed is:

1. A femtosecond laser system for ophthalmology, comprising:
a laser configured to emit a laser beam;
a frequency conversion unit positioned in an optical path of the laser beam and configured to convert the laser beam to a frequency-converted laser beam;
a first sensor configured to measure intensity of the laser beam;
a second sensor configured to measure intensity of the frequency-converted laser beam; and
a processor coupled to each of the first sensor and the second sensor, the processor configured to execute computer-readable instructions to perform or control performance of operations comprising:
receiving measurements of the laser beam and the frequency-converted laser beam from the first and second sensors;
calculating a conversion ratio based on the measurements from the first and second sensors; and
comparing the calculated conversion ratio to a reference ratio to determine whether a performance of the femtosecond laser system has degraded.

2. The femtosecond laser system of claim 1, further comprising a third sensor coupled to the processor and configured to measure intensity of the frequency-converted laser beam downstream of the second sensor.

3. The femtosecond laser system of claim 2, wherein the operations further comprise:
receiving measurements of the frequency-converted laser beam from the third sensor;
calculating a second conversion ratio based on the measurements from the first and third sensors; and
comparing the calculated second conversion ratio to the calculated conversion ratio to determine whether at least one of: the femtosecond laser system has a desired alignment, the second sensor is experiencing degraded performance, or the third sensor is experiencing degraded performance.

4. The femtosecond laser system of claim 1, further comprising an alert system coupled to the processor and configured to generate an alert in response to a determination that the performance of the femtosecond laser system has degraded beyond a first threshold.

5. The femtosecond laser system of claim 4, wherein the operations further comprise automatically powering down the laser in response to a determination that the performance of the femtosecond laser system has degraded beyond a second threshold that is past the first threshold.

6. The femtosecond laser system of claim 1, wherein:
the laser beam comprises infrared radiation;
the frequency-converted laser beam comprises ultraviolet radiation; and
the frequency conversion unit is configured to convert infrared radiation to ultraviolet radiation.

7. The femtosecond laser system of claim 1, wherein the reference ratio is stored in a memory coupled to the processor and is based on initial measurements from the first and second sensors generated at a previous time.

8. The femtosecond laser system of claim 7, wherein the operations further comprise generating a plurality of reference ratios at the previous time, including iteratively setting power of the laser to a different power, and, while at each power:
receiving measurements of the laser beam and the frequency converted laser beam from the first and second sensors while the power of the laser is set at a corresponding power;
calculating a reference ratio for the corresponding power based on the measurements from the first and second sensors; and
storing the reference ratio for the corresponding power in the memory.

9. The femtosecond laser system of claim 1, further comprising a data storage device coupled to the processor, wherein the operations further comprise, each time the femtosecond laser system is used in an ophthalmic procedure, logging at least one of measurements from the first and second sensors or a corresponding calculated conversion ratio in the data storage device.

10. The femtosecond laser system of claim 9, wherein the operations further comprise, analyzing logged data in the data storage device for trend analysis to monitor degradation of the femtosecond laser system over time.

11. A method to monitor parameters of a femtosecond laser system for ophthalmology, the method comprising:
measuring intensity of a laser beam emitted by a laser using a first sensor;
converting the laser beam to a frequency-converted laser beam;
measuring intensity of the frequency-converted laser beam using a second sensor;
calculating a conversion ratio based on the measurements from the first and second sensors; and
comparing the calculated conversion ratio to a reference ratio to determine whether a performance of the femtosecond laser system has degraded.

12. The method of claim 11, further comprising:
measuring intensity of the frequency-converted laser beam using a third sensor downstream of the second sensor;
calculating a second conversion ratio based on the measurements from the first and third sensors; and
comparing the calculated second conversion ratio to the calculated conversion ratio to determine whether at least one of: the femtosecond laser system has a desired alignment, the second sensor is experiencing degraded performance, or the third sensor is experiencing degraded performance.

13. The method of claim 11, further comprising, generating an alert in response to a determination that the performance of the femtosecond laser system has degraded beyond a first threshold.

14. The method of claim 13, further comprising, automatically powering down the laser in response to a determination that the performance of the femtosecond laser system has degraded beyond a second threshold that is past the first threshold.

15. The method of claim 11, wherein converting the laser beam to a frequency-converted laser beam comprises converting infrared radiation to ultraviolet radiation.

16. The method of claim 11, further comprising, at a previous time:
measuring an initial intensity of the laser beam emitted by the laser using the first sensor;
measuring an initial intensity of the frequency-converted laser beam using the second sensor;
calculating an initial conversion ratio based on the measurements of the initial intensity from the first and second sensors; and
storing the initial conversion ratio for future use as the reference ratio.

17. The method of claim 16, further comprising, generating a plurality of reference ratios at the previous time, including iteratively setting power of the laser to a different power, and, while at each power:
receiving measurements of the laser beam and the frequency converted laser beam from the first and second sensors while the power of the laser is set at a corresponding power;
calculating a reference ratio for the corresponding power based on the measurements from the first and second sensors; and
storing the reference ratio for the corresponding power for future use.

18. The method of claim 11, further comprising, each time the femtosecond laser system is used in an ophthalmic procedure, logging at least one of measurements from the first and second sensors or a corresponding calculated conversion ratio.

19. The method of claim 18, further comprising, analyzing logged data for trend analysis to monitor degradation of the femtosecond laser system over time.

20. A non-transitory computer-readable storage medium comprising computer-readable instructions executable by a processor to perform or control performance of operations comprising:
measuring intensity of a laser beam emitted by a laser using a first sensor;
converting the laser beam to a frequency-converted laser beam;
measuring intensity of the frequency-converted laser beam using a second sensor;
calculating a conversion ratio based on the measurements from the first and second sensors; and
comparing the calculated conversion ratio to a reference ratio to determine whether a performance of a femtosecond laser system that includes the laser, the first sensor, and the second sensor has degraded.
